(19) United States
(12) Patent Application Publication
(10) Pub. No.: US 2016/0370288 A1
(43) Pub. Date: Dec. 22, 2016

(54) TRANSIENT SENSING OF AU NANORODS USING TAPERED OPTICAL FIBER

(71) Applicant: Washington University, St. Louis, MO (US)

(72) Inventors: Sahin Kaya Ozdemir, St. Louis, MO (US); Lan Yang, St. Louis, MO (US); Huzeyfe Yilmaz, St. Louis, MO (US)

(21) Appl. No.: 15/161,701

(22) Filed: May 23, 2016

Related U.S. Application Data
(60) Provisional application No. 62/165,954, filed on May 23, 2015.

(51) Int. Cl. G01N 21/552; G01N 33/553; G01N 33/543; G01N 15/14; G01N 21/59

(52) U.S. Cl. CPC G01N 21/554; G01N 15/1436; G01N 21/59; G01N 33/54373; G01N 33/54346; G01N 33/553; G01N 2015/0065

(57) ABSTRACT

A system and method for is transient sensing of Au nanorods at plasmon frequency in aqueous solution using tapered optical fiber.

### CROSS REFERENCE TO RELATED APPLICATION

This Application Claims Priority To and The Benefit Of Provisional Patent Application Ser. No. 62/165,954 Entitled TRANSIENT SENSING OF Au NANORODS USING TAPERED OPTICAL FIBER Filed May 23, 2015

### GOVERNMENT LICENSE RIGHTS STATEMENT

This invention was made with government support under NSF-0954941 awarded by the National Science Foundation; and under ARO W911-NF-12-1-0026 awarded by the Army Research Office. The government has certain rights in the invention.

### BACKGROUND

Field

This technology relates generally to nanoscale transient sensing, and, more particularly, to sensing using tapered optical fibers.

Background Art

Tapered optical fibers have proven to be simple and sensitive platforms for the sensing of single nanoparticles down to sizes of tens of nanometers. In the past, sensing of single nanoparticles with fiber-tapers has relied primarily on the binding of nanoparticles to the optical fiber surface, thereby causing scattering induced reductions in transmitted light intensity. This method limits the lifetime and reusability of the sensor, as the binding of too many nanoparticles to the fiber taper causes a complete loss of transmitted signal.

### BRIEF SUMMARY

The technology as disclosed is transient sensing of Au nanorods at plasmon frequency in aqueous solution using tapered optical fiber. The technology provides nanofiber based sensing system for nanoparticle and biological particle detection and measurement in a liquid environment. The technology provides a method for detecting and measuring nanoparticles and biological particles at single particle resolution in liquid environment. One implementation can include a device that contains a nanofiber, a microfluidic channel, a photodetector and a laser diode. A nanoparticle (including biological particles) can enter the evanescent field of the fiber taper scattering the light leading to change in the transmitted light intensity. The change takes place in two ways. If the particle sits on the nanofiber, discrete changes can be observed in the light intensity. If the particle enters the evanesncent field and carried out by Brownian motion or any other dynamics, the change is reflected as discrete spikes in the detected light intensity. The device and the method works for plasmonic, biological and dielectric nanoparticles. The platform can be configured to detect particle and flow velocities.

Tapered optical fibers have proven to be simple and sensitive platforms for the sensing of single nanoparticles down to sizes of tens of nanometers. In the past, sensing of nanoparticles with fiber-tapers has relied primarily on the binding of nanoparticles to the optical fiber surface, thereby causing scattering induced reductions in transmitted light intensity. This method limits the lifetime and reusability of the sensor, as the adsorption of too many nanoparticles to the fiber-taper causes a complete loss of transmitted signal. We present an aqueous sensing method which overcomes this difficulty by detecting gold nanorods of approximate dimensions 50 nm×20 nm without binding to the fiber-taper. Using laser light near the plasmon frequency of the Au nanorods as our sensing signal, we are able to detect transient drops in transmitted signal intensity when sensing the nanorods in solution. The size and number of these transient events increase as the concentration of nanorods in the sensing solution is increased, indicating that the events are caused by the scattering of light as nanorods enter and subsequently leave the evanescent field around the optical fiber. Our sensing method opens the way to using tapered optical fibers as reusable and long lasting sensors for detecting nanoparticles in solution without the use of micro-cavities.

Industrial Applicability can include:

Nanofiber characterization in liquid environment with visible and near-IR light;

Polystyrene particle detection down to 30 nm with single particle resolution;

Detection of nanogold and nanocages with light closer to and far away from their plasmonic wavelengths;

Discrete jumps and spikes were characterized using statistical analysis as a function of particle size, concentration and polarizability; and

Built a PDMS microfluidic channel for the measurements.

These and other advantageous features of the present invention will be in part apparent and in part pointed out herein below.

### BRIEF DESCRIPTION OF THE DRAWINGS

For a better understanding of the present invention, reference may be made to the accompanying drawings in which:

FIG. 1 is an illustration of nanoparticle around the fiber taper;

FIG. 1A is an illustration of a fiber taper sensing region;

FIG. 2 is an illustration of PS nanoparticles of radius 30 nm sensed in Phosphate Buffered Saline;

FIG. 3 is an illustration of pH induced binding of Au Nanorods to Fiber Taper;

FIG. 4 is an illustration of sensing Au Nanarods at 670 nm Wavelength—200 pM;

FIG. 5 is an illustration of a histogram transient spike event for pM Au nanorod concentrations;

FIG. 6 is an illustration of one configuration under test;

FIG. 7A is an illustration of a graph showing sensing of Au Nanorods at 670 nm Wavelength: opM;

FIG. 7B is an illustration of a graph showing sensing of Au Nanorods at 670 nm wavelength: 200 pM;

FIG. 8 is an illustration of a graph showing a change in signal upon flushing chamber;

FIG. 9 is an illustration of a graph showing a histogram spike height before and after flushing sensor;

FIG. 10 is an illustration of a graph showing Au Nanorod sensing with 665 nm light—solution with pH approximately 1-2;

FIG. 11 is another illustration of a graph showing Au Nanorod sensing with 665 nm light—Detail;

FIG. 12 is an illustration of a graph showing a distribution of events; and

FIGS. 13A and 13B are an illustration of SEM images shoing AuNR absorbed on Silica Fiber-Taper.

While the invention is susceptible to various modifications and alternative forms, specific embodiments thereof are shown by way of example in the drawings and will herein be described in detail. It should be understood, however, that the drawings and detailed description presented herein are not intended to limit the invention to the particular embodiment disclosed, but on the contrary, the intention is to cover all modifications, equivalents, and alternatives falling within the spirit and scope of the present invention as defined by the appended claims.

### DETAILED DESCRIPTION OF INVENTION

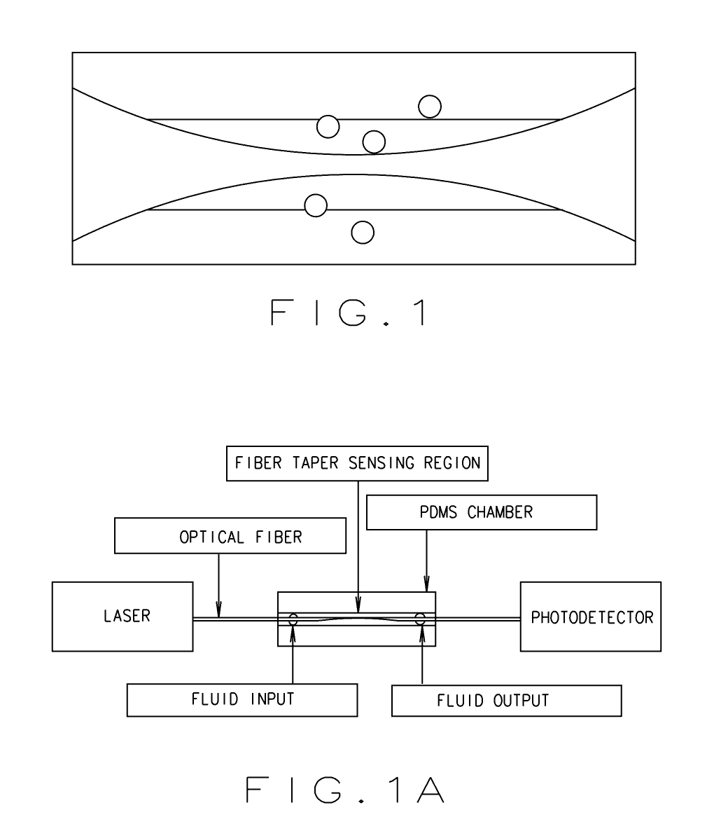

According to the embodiment(s) of the present invention, various views are illustrated in FIG. 1-13 and like reference numerals are being used consistently throughout to refer to like and corresponding parts of the invention for all of the various views and figures of the drawing. Also, please note that the first digit(s) of the reference number for a given item or part of the invention should correspond to the Fig. number in which the item or part is first identified.

One implementation of the present technology teaches an aqueous sensing system and method for detecting gold nanorods of approximate dimensions 50 nm×20 nm without binding to the fiber-taper. The technology uses laser light near the plasmon frequency of the Au nanorods as a sensing signal, such that the technology is able to detect transient drops in transmitted signal intensity when sensing the nanorods in solution. The size and number of these transient events increase as the concentration of nanorods in the sensing solution is increased, indicating that the events are caused by the scattering of light as nanorods enter and subsequently leave the evanescent field around the optical fiber. The sensing method opens the way to using tapered optical fibers as reusable and long lasting sensors for determining the concentration or size of nanoparticles in solution.

Tapered optical fiber sensors consist of a portion of optical fiber that has been tapered, usually by heating and pulling, to diameters ranging from micron to nanometer scale. This tapering removes the protective effect of the fiber cladding, allowing an evanescent field to propagate through the medium surrounding the fiber taper. For a single-mode fiber, as the taper diameter is decreased, the effective refractive index of the taper will approach a value close to the refractive index of the cladding, as the cladding makes up a larger portion of the fiber than the core. The evanescent field surrounding the taper will decay as:

E(r,t)=E₀(r,t)e^{-αρ}

Where α is the decay parameter and ρ is the distance from the taper medium interface. The decay parameter α is given by:

$$α = \frac{2 π}{λ} \sqrt{{n}_{fiber}^{2} {sin}^{2} {θ}_{i} - {n}_{medium}^{2}}$$

Where λ is the wavelength of guided light and is the angle of light incidence at the taper medium boundary. Thus, the depth that the evanescent field penetrates into the surrounding medium will be proportional to the wavelength of the light. Longer wavelengths of light will penetrate farther into the medium, while shorter wavelengths will create a more localized, and hence more sensitive evanescent field. Tapered optical fibers have been used for a number of applications, including bulk sensing of biomolecules, DNA hybridization sensing, magnetic field sensing, refractive index sensing, and single nanoparticle detection and sizing. Most sensing schemes using tapered optical fibers rely on one of two effects to sense the desired material. The first scheme monitors the change in transmitted intensity of a single wavelength of light guided through the taper as the sensing analyte interacts with the evanescent field. This change in intensity can be caused by bulk effective refractive index change of the surrounding medium upon introduction of the analyte or can be caused by scattering or absorption caused by the analyte.

The second scheme involves scanning the wavelength and monitoring the change in transmission intensity vs wavelength as the analyte is introduced. The advantage of the first (single wavelength) scheme over the second is that it allows for the detection of single particles down to the nanoscale. It has been demonstrated that tapered optical fiber sensors can be used not only to detect single nanoparticles down to radii of 120 nm, but also to size them. Their detection method relies on the premise that as nanoparticles bind to the fiber taper and interact with the evanescent field, they will scatter light and decrease the transmitted light intensity (FIG. 1). The drop in intensity will appear as a discrete step in the transmitted signal over time. If the nanoparticles are spherical dielectrics, then they may be modeled as Rayleigh scatterers and the size of the intensity step will obey the relation:

h∝α²∝R⁶

Here, α represents the polarizability of a given nanoparticle, and the radius of the nanoparticle. Thus, a nanoparticle can be sized by observing the size of intensity step that occurs when the nanoparticle binds to the taper.

The details of the invention and various embodiments can be better understood by referring to the figures of the drawing. Referring to FIG. 1, an illustration of nanoparticles around the fiber taper is provided.

The experiments to demonstrate the technology were conducted with the fiber taper in air and the nanoparticles were launched toward the taper using an atomizer and differential mobility analyzer. While these results are promising, it is interesting to consider extending similar experiments to aqueous environments. Aqueous settings are common in many potential applications, including in the biomedical domain. The ability to sense nanoparticles in aqueous solution can open up a wide range of applications. Detection of radius 100 nm polystyrene nanoparticles as well as gold nanorods of approximate dimensions 40×16 nm in aqueous solution has been demonstrated using a pair of tapered optical fibers. The technology demonstrated detection of single spherical polystyrene nanoparticles of radius 30 nm in phosphate buffered saline (PBS) solution using the transmission step method of Zhu et. al described above and 670 nm light (FIG. 2). FIG. 1A at the end of this document shows a simple schematic of the experimental setup. Single-mode SM600 fiber was tapered to a single mode diameter of 500 nm. These results extend the results of Zhu et. al into the aqueous domain, demonstrating that tapered optical fibers possess the sensitivity to detect single nanoparticles in solution and setting a new small-size detection record.

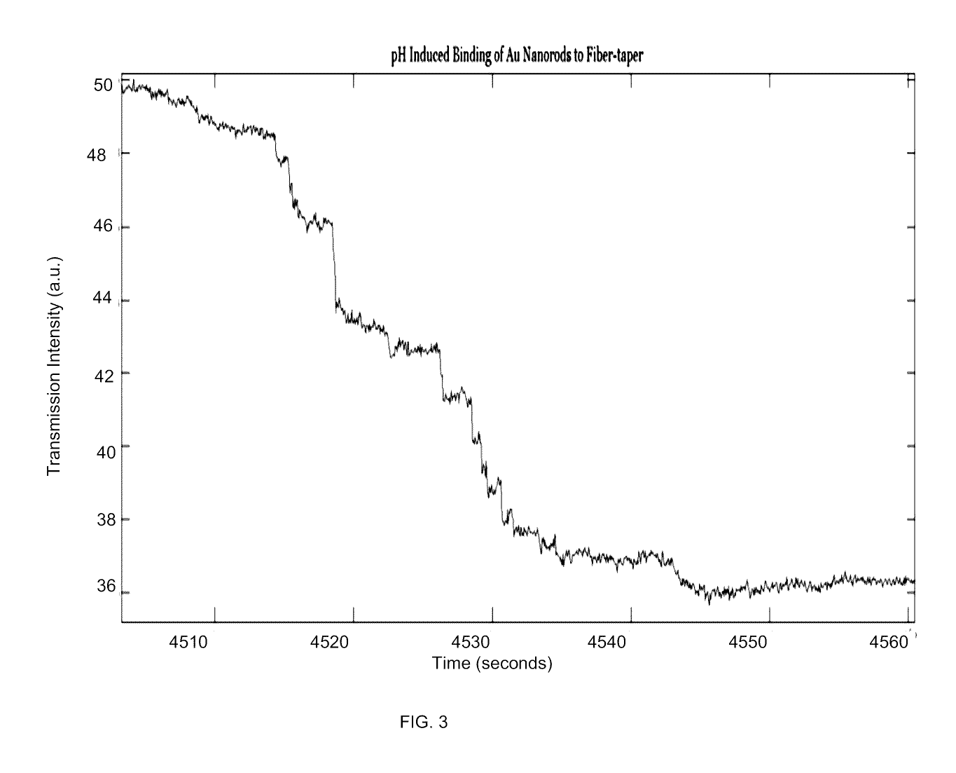

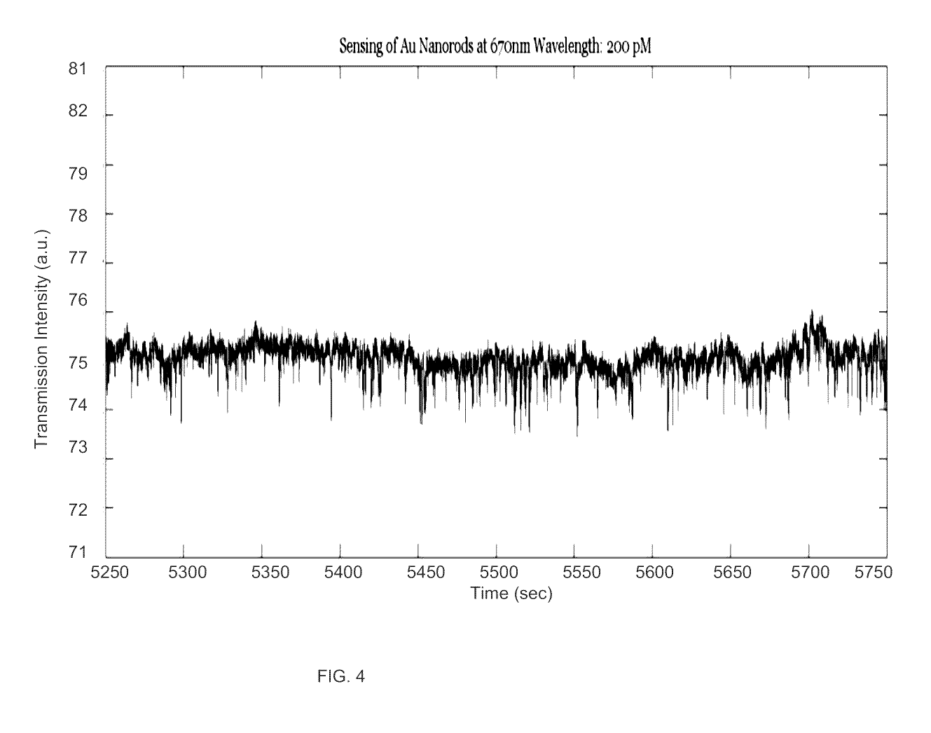

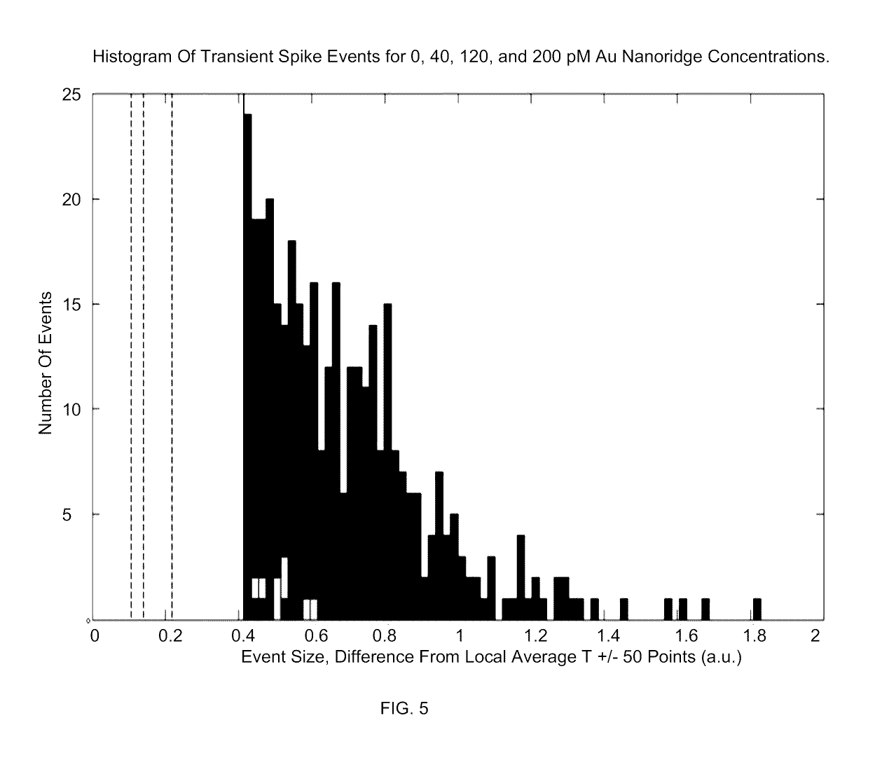

Experiments were conducted demonstrating detection of single Au nanorods of approximate dimensions 50×20 nm and a longitudinal plasmon resonance at 610 nm synthesized in a Soft Nanomaterials Laboratory. As in the experiments involving polystyrene nanoparticles, an SM600 single-mode tapered fiber was used with a sensing wavelength of 670 nm. When the experiments were conducted in an HCl/water solution of pH approximately two, the nanorods bound to the fiber-taper, causing discrete drops in transmission intensity similar to the results for polystyrene nanoparticles (FIG. 3). However, when the experiments were conducted in DI water, the Au nanorods were unable to bind to the fiber-taper. Instead, transient drops, or downward spikes, in the transmission intensity were observed. A representative signal is visible in FIG. 4. As the number of nanorods added to the solution was increased, so too did the number and size of spikes. The number of spikes over equal time intervals was measured for several concentrations of Au nanorods (FIG. 5). The distributions of transient spikes collected over 500 seconds for several different concentrations of AuNR in the sensing chamber are shown at left. The histogram is truncated at the 3σ noise level. There is no significant difference between the distribution for 40 pM and the baseline 0 pM. There is a significant difference between the distributions for 120 pM and 200 pM and the baseline, indicating the presence of AuNR in the solution.

These results demonstrate a new method for sensing nanoparticles using optical fiber tapers that relies on transient events, rather than semi-permanent binding, to sense nanoparticles. The advantage of this method is that transmission intensity is maintained throughout sensing, meaning that the baseline signal is recoverable. The technology as disclosed has been confirmed experimentally by first observing a signal from a large concentration of Au nanorods and then flushing the solution with clean DI water. After flushing, the baseline signal was regained. These results indicate that fiber taper sensors can be used to sense nanoparticles in a way that renders the sensor reusable, which is crucial if tapered optical fibers are to be used in engineering applications. Many possibilities for further experiments with fiber-tapers exist. Results indicate the strong capabilities of tapered optical fibers to sense, both transiently and through binding, several kinds of nanoparticles. These results can be used as a basis for conducting further experiments and devising important applications. With the appropriate choice of surface chemistry, the kind of nanoparticle or molecule that binds to the silica fiber-taper can be carefully controlled. This opens the way for sensor selectivity in environments containing a number of different species, such as in biological settings. In addition, nanoparticles such as the Au nanorods that we tested can be conjugated to biomolecules of interest, in principle allowing for tagging and subsequent transient detection of biomolecules.

Also promising is the enhancement of sensor sensitivity using surface plasmon resonance. Surface plasmon enhancement using Au nanoparticles bound to a tapered optical fiber surface has already been demonstrated, but only for bulk refractive index change measurements. Recently, plasmonic enhanced sensing of the interactions of single 8-mer oligonucleotides has been demonstrated using Au nanorods bound to silica microspheres. Similar techniques could be applied to optical fiber-tapers in order to allow for sensing of single biomolecules. A key advantage of tapered optical fibers over silica microspheres, especially for biological applications, is comparative simplicity and small size. If sensing results for single biomolecules can be extended to tapered optical fibers, the implications will be significant for future applications in biomedicine. A utility can be the use of tapered optical fiber technology as disclosed to sense cationic replacement reactions in metal nanoparticles.

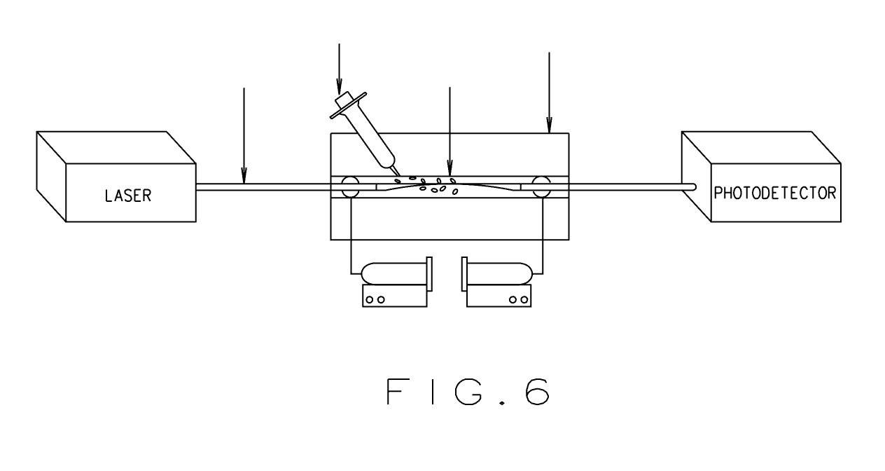

Referring to FIG. 6 an illustration of one implementation of the Technology under test is shown. In all experiments, single-mode SM600 silica optical fiber was tapered using a hydrogen flame to single-mode taper diameter (approx. 500 nm for light in the range 665 nm to 675 nm). The fiber-taper was placed inside a micro-fluidic PDMS chamber with a total volume of 0.24 mL to hold the aqueous solution to which AuNR are added. Programmable micro-fluidic pumps were used to pump solution through the chamber via the fluid input/output ports. A laser, tunable in the range 665 nm to 675 nm, provided a constant wavelength sensing signal which was coupled into the optical fiber. A photodetector was used to to monitor changes in the intensity of the signal transmitted through the tapered fiber. The gold nanorods (AuNR) tested were synthesized in the Washington University Soft Nanomaterials lab, have a longitudinal plasmon resonance centered at 610 nm, and have approximate dimensions 50×20 nm. See, L. Tian, E. Chen, N. Gandra, A. Abbas, and S. Singamaneni, “Gold Nanorods as Plasmonic Nanotransducers: Distance-Dependent Refractive Index Sensitivity,” Langmuir, 28, 17435-17442, 2012. Transient sensing experiments were conducted in DI water and binding experiments were conducted in DI water with pH reduced to approx. 1-2 with hydrochloric acid.

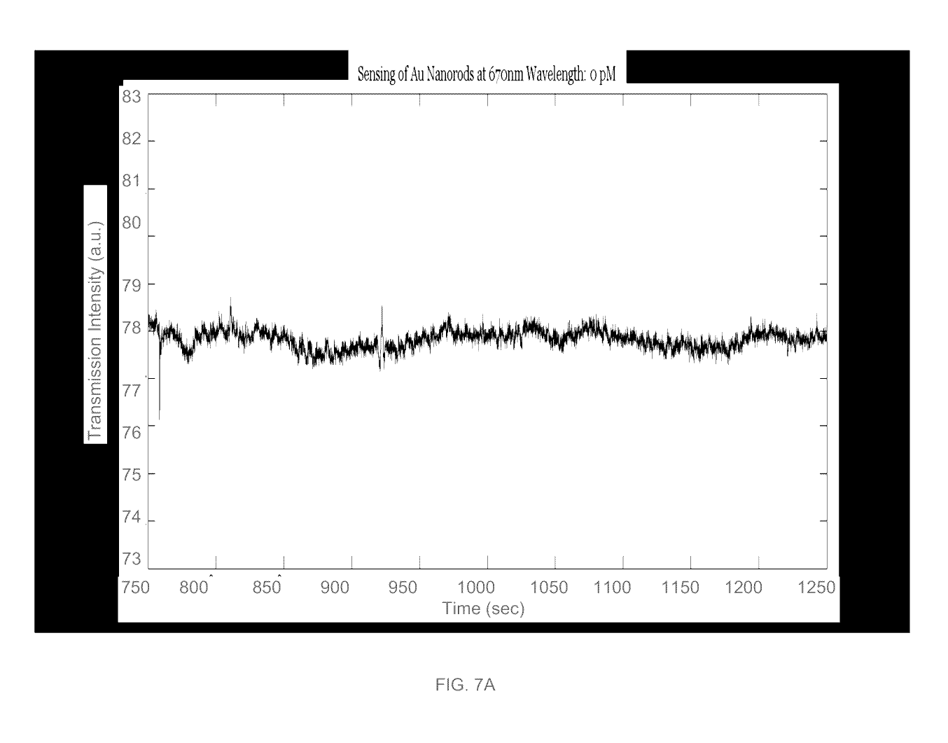

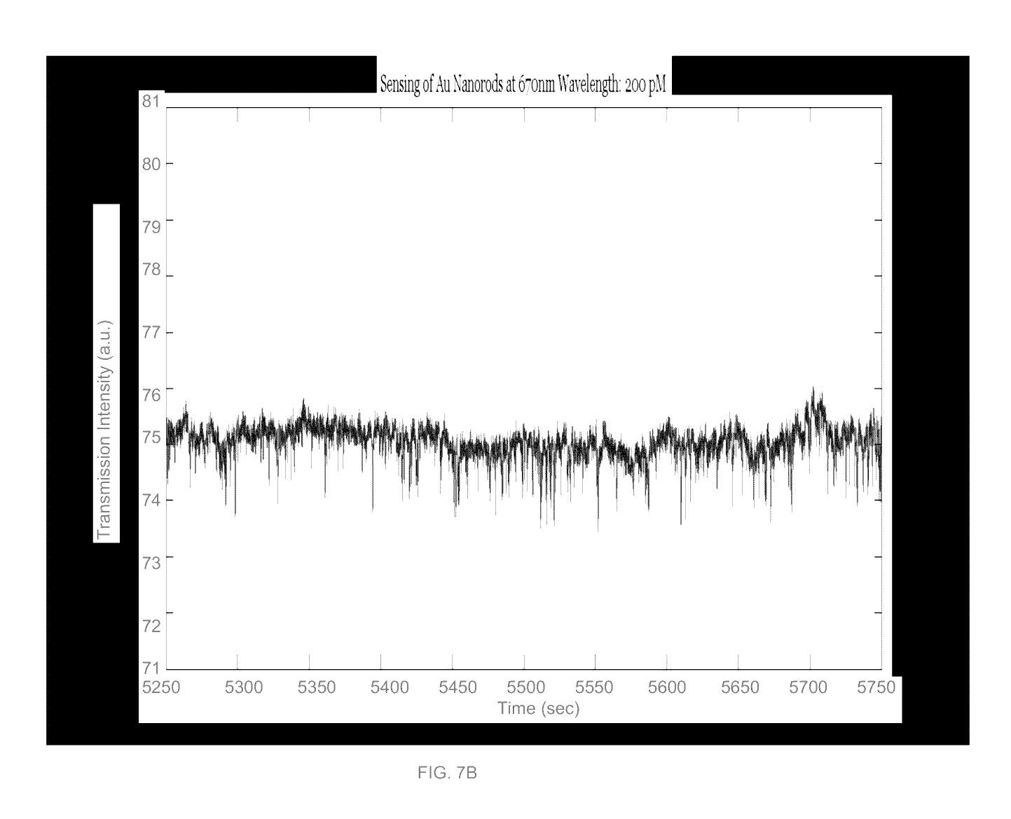

Referring to FIGS. 7A and 7B Transient Gold Nanorod Sensing Gold Nanorod Binding is shown with an illustration of a graph showing sensing of Au Nanorods at 670 nm Wavelength: opM; and an illustration of a graph showing sensing of Au Nanorods at 670 nm wavelength: 200 pM are shown respectively. FIG. 7A is the baseline signal—no Nanorods present. In DI water, AuNR do not bind to the silica fiber-taper. When no AuNR are present in the chamber solution, the transmitted signal is stable—occasional transient downward spikes are present due to solution impurities. AuNR were added to the chamber using a micropipette and allowed to diffuse. Upon the introduction of AuNR to the chamber, a significant number of transient events are visible as downward spikes in the transmitted signal. A higher concentration of AuNR yields larger and more numerous spikes. These results indicate that the transient events occur when AuNR pass near the taper and interact strongly with the surrounding evanescent field, causing an instantaneous reduction in the transmitted light intensity.

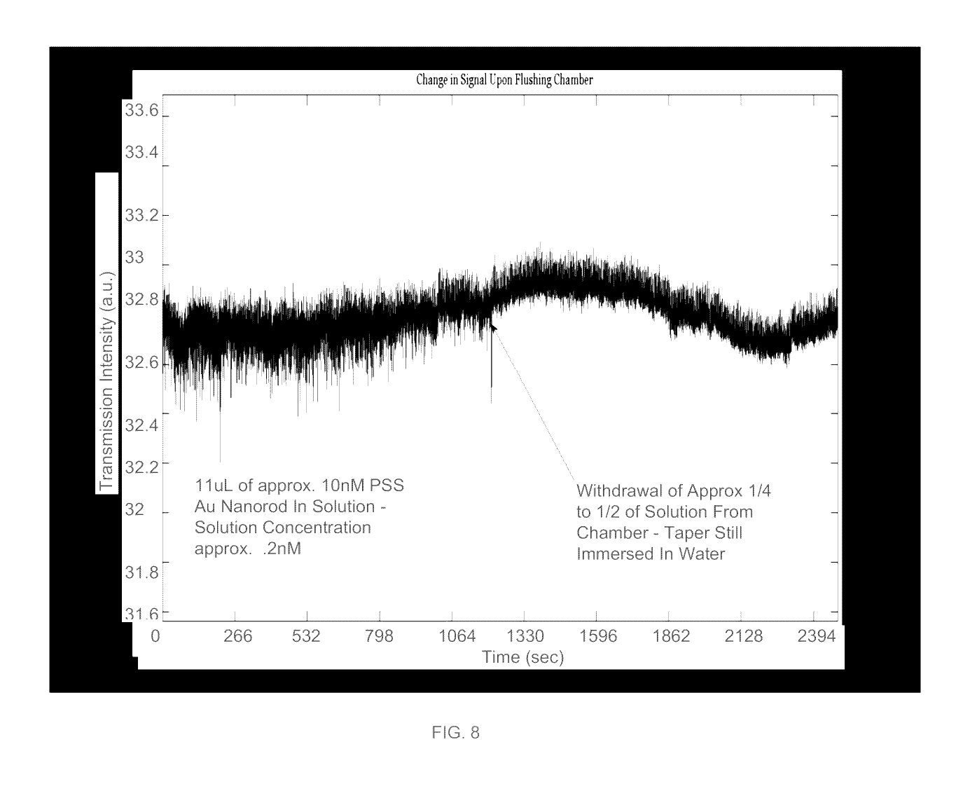

Referring to FIG. 8 and FIG. 9 an illustration of a graph showing a change in signal upon flushing chamber; and an illustration of a graph showing a histogram spike height before and after flushing sensor is shown respectively. The sensing signal from a 200 pM concentration of AuNR is shown at left. Upon flushing of the solution chamber, a dramatic reduction in the number of transient events (downward spikes) is evident, and a return to the baseline signal is seen. The difference in fluctuations from the local average of the signal before and after flushing can be seen at right. These results demonstrate the reusability of the tapered-fiber nanoparticle sensor when used in an aqueous setting to transiently sense nanoparticles. As indicated by the arrow—Withdrawal of Approx. ¼ to ½ of Solution from Chamber—Taper Still Immersed in Water.

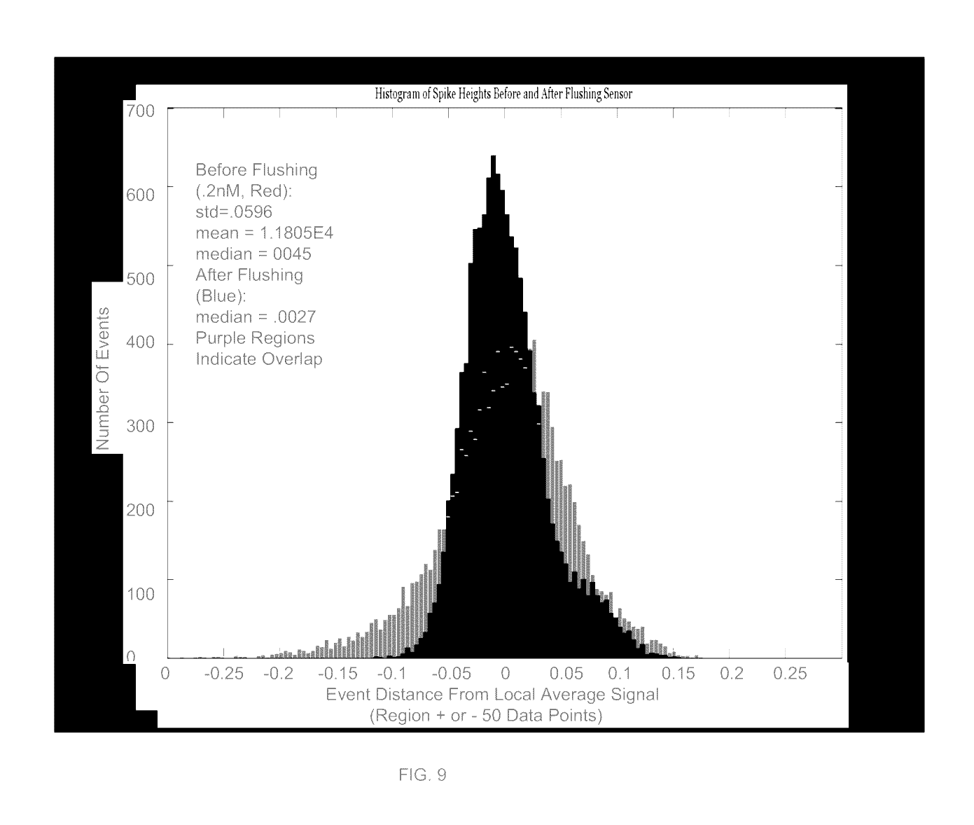

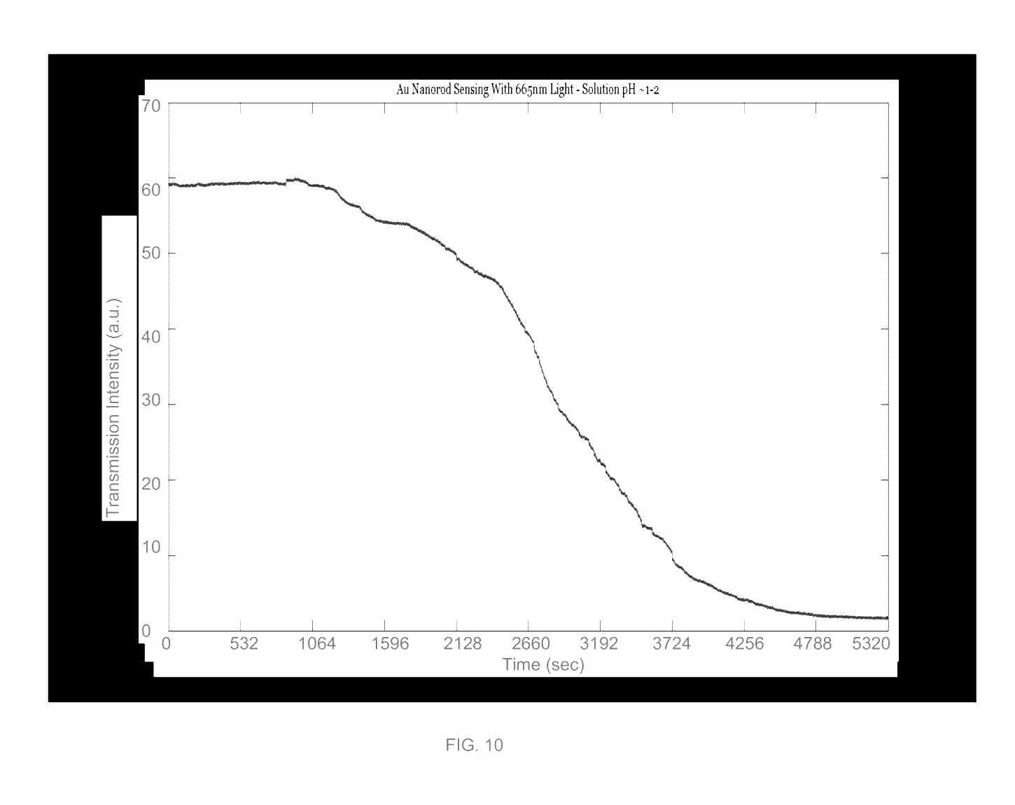

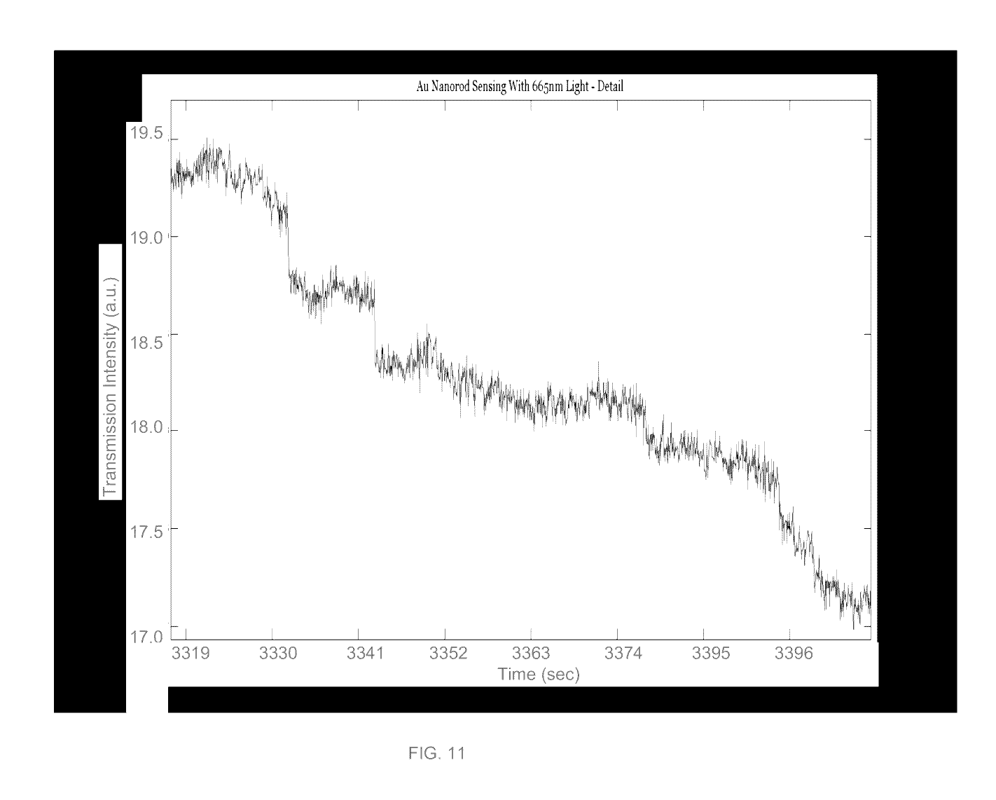

Gold Nanorod binding is illustrated. Referring to FIG. 10 an illustration of a graph showing Au Nanorod sensing with 665 nm light—solution with pH approximately 1-2 is provided; FIG. 11 where another illustration of a graph showing Au Nanorod sensing with 665 nm light—Detail is provided; and FIG. 12 where an illustration of a graph showing a distribution of events is provided. Upon reducing the pH of the chamber solution with HCl to ˜1-2, binding of AuNR to the silica fibertaper will occur. See M. D. Baaske, M. R. Foreman, and F. Vollmer, “Single-molecule nucleic acid interactions monitored on a label-free microcavity biosensor platform,” Nature Nanotechnology, 9, 933-939, 2014. Transient events are no longer present. Instead, discrete drops in the transmitted signal can be seen, corresponding to AuNR binding events. The size of each binding event can be measured by performing a running cross correlation of the data with a step-function. For ideal spherical nanoparticles, modeled as Rayleigh scatterers, the size of each event follows the relation. See, J. Zhu, S. K. Özdemir, and L. Yang, “Optical Detection of Single Nanoparticles With a Subwavelength Fiber-Taper,” IEEE Photonics Technology Letters, vol. 23, 1346-1348, 2011.

Similarly, for a gold nanorod, modeled as an ellipsoid of semimajor axis and semi-minor axes and , the size of each event follows the relation. J. Zhu, L. Huang, J. Zhao, Y. Wang, Y. Zhao, L. Hao, and Y. Lu, “Shape dependent resonance light scattering properties of gold nanorods,” Materials Science and Engineering B, vol. 121, 199-203, 2005. Thus, by plotting the distribution of events , we achieve the distribution at left of events with units of length. This distribution is characteristic of a single size of Au nanoparticle, as expected. After conducting a AuNR binding experiment, the fiber-taper was removed from the PDMS chamber and taken to a scanning electron microscope for imaging. AuNR are clearly visible on the tapered fiber, validating our results. These results indicate that these AuNR can still be detected and sized using established immobilization methods for nanoparticle detection using fibertaper and that solution pH is the determining parameter for transient or irreversible detection.

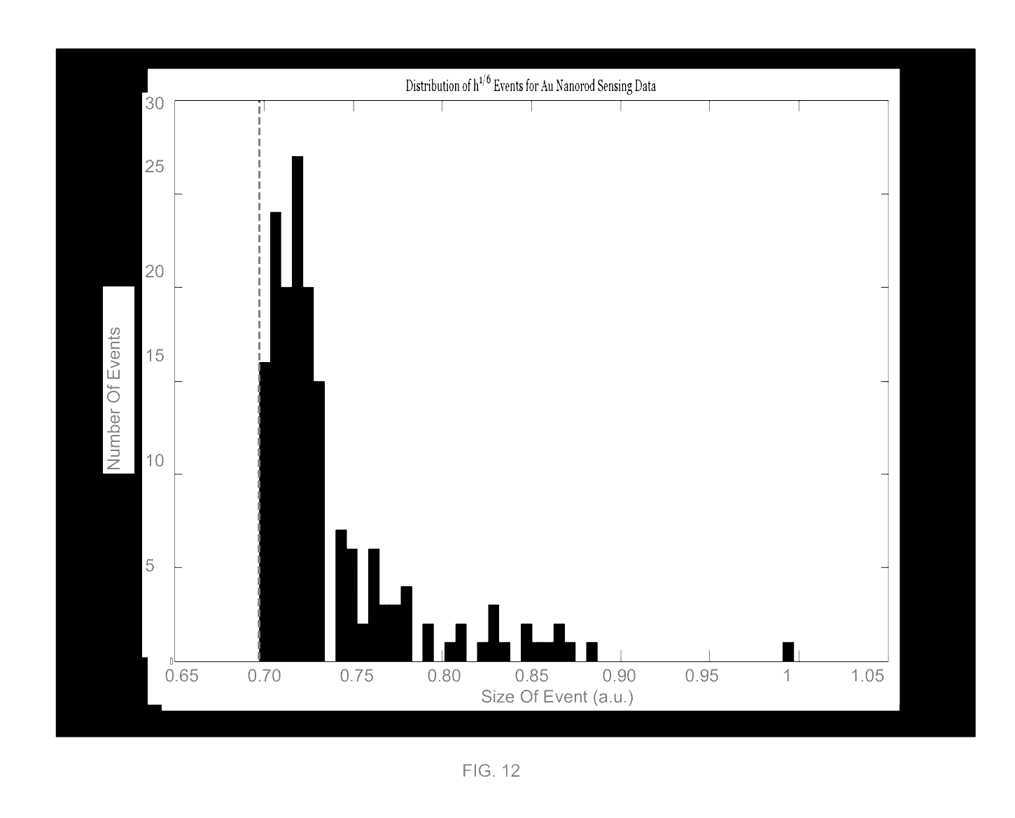

FIGS. 13A and 13B are SEM Images Showing AuNR Adsorbed on Silica Fiber-Taper.

The various examples shown above illustrate the technology as disclosed. A user of the present technology may choose any of the above implementations, or an equivalent thereof, depending upon the desired application. In this regard, it is recognized that various forms of the subject technology as disclosed could be utilized without departing from the spirit and scope of the present invention.

As is evident from the foregoing description, certain aspects of the present invention are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the sprit and scope of the present invention.

Other aspects, objects and advantages of the present invention can be obtained from a study of the drawings, the disclosure and the appended claims.

### REFERENCES

1. Ravets, S., Hoffman, J. E., Kordell, P. R., Wong-Campos, J. D., Rolston, S. L., & Orozco, L. A. (2013). Intermodal energy transfer in a tapered optical fiber: optimizing transmission. Journal of the Optical Society of America A, 2361-2371.

2. Palais, J. (1988). Fiber optic communications (2nd ed.). Englewood Cliffs, N.J.: Prentice Hall.

3. Tian, Y., Wang, W., Wu, N., Zou, X., & Wang, X. (2011). Tapered Optical Fiber Sensor for Label-Free Detection of Biomolecules. Sensors, 3780-3790.

4. Leung, A., Shankar, P., & Mutharasan, R. (2008). Label-free detection of DNA hybridization using gold-coated tapered fiber optic biosensors (TFOBS) in a flow cell at 1310 nm and 1550 nm. Sensors and Actuators B, 131.

5. Ji, W., Tjin, S., Lin, B., & Ng, C. (2013). Highly Sensitive Refractive Index Sensor Based on Adiabatically Tapered Microfiber Long Period Gratings. Sensors, 14055-14063.

6. Deng, M., Liu, D., & Li, D. (2014). Magnetic field sensor based on asymmetric optical fiber taper and magnetic fluid. Sensors and Actuators A: Physical, 55-59.

7. Zhu, J., Ozdemir, S. K., & Yang, L. (2011). Optical Detection of Single Nanoparticles with a Sub-wavelength Fiber-Taper. Photonics Technology Letters, IEEE, 23(18).

8. Yu, X. C., Li, B. B., Wang, P., Tong, L., Jiang, X. F., Li, Y., Gong, Q., Xiao, Y. F. (2014). Single Nanoparticle Detection and Sizing Using a Nanofiber Pair in an Aqueous Environment. Advanced Materials, 26, 7462-7467.

9. Tian, L., Chen, E., Gandra, N., Abbas, A., & Singamaneni, S. (2012). Gold Nanorods as Plasmonic Nanotransducers: Distance-Dependent Refractive Index Sensitivity. Langmuir, 17435-17442.

10. Tamerler, C., Kacar, T., Sahin, D., Fong, H., & Sarikaya, M. (2007). Genetically engineered polypeptides for inorganics: A utility in biological materials science and engineering. Materials Science and Engineering C, 558-564.

11. Joshi, P., Yoon, S. J., Hardin, W., Emelianov, S., & Sokolov, K. (2013). Conjugation of antibodies to gold nanorods through Fc portion: synthesis and molecular specific imaging. Bioconjug Chem., 878-888.

12. Lin, H., Huang, C., Cheng, G., Chen, N., & Chui, H. (2012). Tapered optical fiber sensor based on localized surface plasmon resonance. Optics Express, 21693-21693. pg. 16

13. Baaske, M., Foreman, M., & Vollmer, F. (2014). Single-molecule nucleic acid interactions monitored on a label-free microcavity biosensor platform. Nature Nanotechnology, 933-939

What is claimed is:

1. A nanofiber based sensing system comprising:
a transient sensing sensor including a tapered optical fiber in an aqueous solution configured to sense at a plasmon frequency nano particles entering a tapered region having an evanesncent field of the tapered optical fiber.
